(19) United States
(12) Patent Application Publication
(10) Pub. No.: US 2008/0045457 A1
(43) Pub. Date: Feb. 21, 2008

(54) Ancestral Hepatitis C virus envelope protein sequence

(76) Inventors: Xiaofeng Fan, Saint Louis, MO (US); Adrian Michael Di Bisceglie, Ladue, MO (US)

Correspondence Address: SAINT LOUIS UNIVERSITY;OFFICE OF INNOVATION AND INTELLECTUAL PROPERTY, 3556 CAROLINE MALL, SUITE C208, ST. LOUIS, MO, 63104, US

(21) Appl. No.: 11/809,365

(22) Filed: May 31, 2007

Related U.S. Application Data
(60) Provisional application No. 60/812,001, filed on Jun. 8, 2006.

(51) Int. Cl. A61K 38/16; A61K 31/711; C07H 19/04; C07K 14/00; C07K 2/00; C07K 7/06; C07K 7/08; C12N 15/63

(52) U.S. Cl. 514/12; 435/320.1; 514/44; 530/300; 530/324; 530/326; 530/328; 530/350; 536/23.72

(57) ABSTRACT

The present invention provides ancestral sequences for hepatitis C virus (HCV) envelope proteins, as well as uses for such sequences in inducing protective immune responses in subjects infected or at risk of infection with HCV.

### PRIORITY CLAIM

This application for patent claims priority to U.S. provisional patent application No. 60/812,001, which was filed on 8 Jun. 2006.

### BACKGROUND OF THE INVENTION

A. Field of the Invention

The invention is directed to nucleic acid and polynucleotide sequences useful in the development of vaccines. The invention is directed to nucleic acid and polypeptide sequences of a hypothetical ancestral Hepatitis C Virus (HCV) envelope proteins and methods for deriving said sequences.

B. Description of the Related Art

HCV, first cloned and sequenced in 1989, is an enveloped single-stranded, positive-sense RNA virus classified into the Flaviviridae family (Choo et al., 1989). HCV has been well-documented as the major etiological agent responsible for most post-transfusional and community-acquired hepatitis (Alter et al., 1999). HCV results in persistent infection in up to 80% of infected individuals and causes a wide spectrum of liver diseases, including cirrhosis and hepatocellular carcinoma (Di Bisceglie, 1998). HCV-related liver disease is now the leading cause of liver transplantation in the United States. The Centers for Disease Control and Prevention have estimated that HCV causes 8,000˜10,000 deaths each year, with deaths expected to more than triple over the next two decades, eventually exceeding those from acquired immunodeficiency syndromes (Natl. Institutes of Health Consensus Dev. Conf. Panel, 1997).

There is no HCV vaccine available currently, with genetic variability and impaired adaptive immunity being the two major obstacles in the development of such reagents. Furthermore, due to the potential for infection with more than one of the six different HCV groups, an effective HCV vaccine should also induce a strong protective response against all groups for a sustained period, placing an additional constraint in development.

### SUMMARY OF THE INVENTION

The inventor has derived certain polynucleotide and polypeptide sequences that represent conceptual ancestral sequences of the envelope proteins of at least the six major HCV groups. The inventor envisions that any one or more of the sequences can be used as an effective multivalent vaccine directed against all HCVs.

In one embodiment, the invention is directed to a conceptually-derived ancestral HCV envelope protein and polynucleotide, which represents a hypothetical ancestor for the current six HCV genotypes (SEQ ID NOS:1 and 2). In addition, conceptually-derived ancestral HCV envelope protein and polynucleotide sequences are provided for the HCV genotype 1 group (SEQ ID NOS:3 and 4). Codon optimized polynucleotides are provide as SEQ ID NO:5 and 6. Polynucleotide sequences having at least 81%, 84%, 86%, or 88% identical to SEQ ID NO:1 or SEQ ID NO:3 also are provided

In yet another embodiment, the invention is directed to a vaccine comprising an ancestral HCV sequence (supra), wherein the vaccine protects a recipient of the vaccine against all six HCV groups. In another embodiment, the invention is directed to an immune system stimulating composition comprising an ancestral HCV sequence (supra), wherein the composition elicits an immune reaction in the recipient against all six HCV groups.

In particular embodiments, the present invention is direct to a polynucleotide comprising a sequence that encodes a polypeptide comprising a sequence as set forth in SEQ ID NOS:2 or 4, or more specifically, comprising the nucleotide sequence as set forth in SEQ ID NOS:1, 3, 5 or 6. The polynucleotide may also consist essentially of a sequence that encodes a polypeptide comprising a sequence as set forth in SEQ ID NOS: 2 or 4, or more specifically, consisting essentially of the nucleotide sequence as set forth in SEQ ID NOS:1, 3, 5 or 6. In addition, applicants contemplate related sequences that are greater than 81% identical to SEQ ID NOS: 1, 3, 5, or 6, including those that are greater than 84%, 86% or 88% identical to SEQ ID NOS: 1, 3, 5 or 6. Similarly, applicants contemplate related sequences that are greater than 81% identical to SEQ ID NOS: 2 or 4, including those that are greater than 84%, 86% or 88% identical to SEQ ID NOS: 2 or 4.

In another embodiment, there is provided a polypeptide comprising the amino acid sequence of SEQ ID NOS: 2 or 4. Also contemplated are fragments of SEQ ID NOS: 2 or 4 that comprise epitopes capable of eliciting an immune response against 2, 3, 4, 5 or all 6 HCV groups. For example, the fragment may be a peptide comprising 10 to about 40 consecutive residues of SEQ ID NO:2 or 4, such as 10, 15, 20, 30 or 40 consecutive residues of SEQ ID NO:2 or 4. The fragments and full length polypeptides may, in some embodiments, be fused to other non-HCV peptide or polypeptide sequences, such as carrier polypeptides, immune modulators, targeting sequences, etc.

Yet another embodiment provides an expression vector that comprises a polynucleotide as described above. In particular, the expression vector encodes a polypeptide comprising a sequence as set forth in SEQ ID NOS: 2 or 4, wherein said polynucleotide is operably connected to a promoter. The nucleotide sequence may be as set forth in SEQ ID NOS:1, 3, 5, or 6. The expression vector may be a non-viral construct or viral construct, such as an adenoviral construct.

In still another embodiment, there is provided a method of inducing an immune response in a subject comprising administering to said subject a composition comprising a polypeptide comprising the amino acid sequence of SEQ ID NOS: 2 or 4, or an immunogenic fragment thereof. The subject may be at risk of contracting HCV, and the subject may be a human. The composition may further comprise an adjuvant. The method may further comprise administering said composition to said subject a second time. The method may also further comprise assessing the immune response is said subject following administering.

In a further embodiment, there is provided a method of inducing an immune response in a subject comprising administering to said subject a composition comprising an expression construct comprising a polynucleotide encoding a polypeptide comprising the amino acid sequence of SEQ ID NOS: 2 or 4, or an immunogenic fragment thereof, wherein said polynucleotide is under the control of a promoter active in cells of said subject. The expression construct may be a non-viral or viral (e.g., adenoviral) expression construct.

As used herein the specification, “a” or “an” may mean one or more. As used herein in the claim(s), when used in conjunction with the word “comprising”, the words “a” or “an” may mean one or more than one. As used herein “another” may mean at least a second or more. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1. Organization of the HCV genome. The 5′-UTR and 3′-UTR are shown as lines. Between them is an open reading frame encoding a single polyprotein processed into structural or non-structural proteins as shown in boxes. Nucleotide numbering is according to HCV J4 strain, GenBank accession number D10749.

FIG. 2. A simple example to elucidate the concept of ancestral sequences. Evolutionary relationship among five current HCV sequences is established by the construction of a phylogenetic tree that connects these sequences through a common node, i.e., most recent common ancestor (MRCA) of HCV genotype 1 isolates. At this point HCV genotype 1 starts diversifying into three subtypes, 1a, 1b and 1c.

FIG. 3. Maximum likelihood reconstruction with 119 full-length HCV envelope sequences (each 1665 bp). All genotypes are indicated. Bootstrap test was done with 100 replicates as shown at major branches. The tree was rooted by applying molecular clock. The nodes at which the ancestral sequences will be inferred are also indicated. MRCA, most recent common ancestor.

FIG. 4. Similarity plot of HCV Chiron strain (GenBank Accession number M62321) to each ancestral HCV envelope sequences.

### DETAILED DESCRIPTION OF THE INVENTION

The present invention relates to the derivation of ancestral sequences for hepatitis C virus (HCV). Such sequences can be used in the development of a pluripotent vaccine that is capable of generating protective immunity against the multiple different HCV genotype groups.

### I. HCV AND PROTECTIVE IMMUNITY

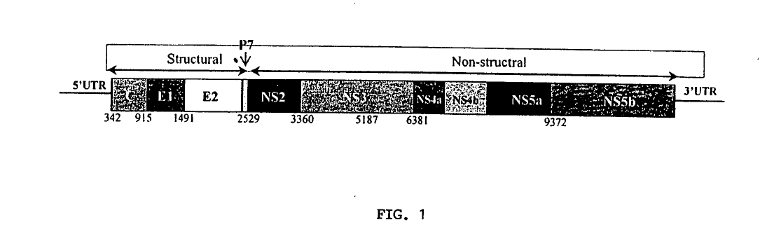

The HCV virion contains a lipid envelope studded with viral envelope proteins that surrounds a protein capsid. Within the capsid is the viral genome comprised of a ˜9600 bp RNA molecule which is divided into three regions (FIG. 1). The 5′-untranslated region (UTR) of the genome is highly structured and contains an internal ribosome entry site that permits efficient translation from the uncapped RNA genome. The 5′-UTR is followed by a single large open reading frame that encodes a single polyprotein of approximately 3010 amino acids. This polyprotein is processed into at least 10 functional proteins by host and viral proteases (Blight and Rice, 1997). Finally, there is a 200˜300 bp 3′-UTR composed of a variable sequence of about 40 bp, a polyU sequence of variable length, a polypyrimidine tract, and a high conserved 98 bp terminal sequence that folds into a highly conserved structure (Kolykhalov et al., 1996; Yamada et al., 1996).

The HCV genome is highly variable. Based on the phylogenetic analysis of nucleotide sequences, HCV is divided into at least six major genotypes (20-30% sequence difference) and more than 50 subtypes (10-20% sequence difference) (Robertson et al., 1998). Moreover, even within an individual infected with a single HCV subtype, HCV circulates as a group of different but genetically closely related variants, referred to as viral quasi-species (less than 10% sequence difference), a characteristic shared by most of RNA viruses (Eigen, 1993). The molecular basis for HCV quasi-species nature is the high viral turn-over rate (about 10¹² virions per day) (Neumann et al., 1998) and the high error rate of its RNA-dependent RNA polymerase encoded by HCV NS5B, which lacks proof-reading repair activity (Holland et al., 1982). Although the variability has been well documented across the entire HCV genome (Simmonds, 1995), the most variable regions are located on envelope domains. In particular, the 5′ end of the second envelope sequence, an 81 bp domain, has been proved to be extremely variable, named hypervariable region 1 (HVR1) (Hijikata et al., 1991; Kato et al., 1992).

Genetic variability has multiple implications for HCV pathogenesis and vaccine development. First, it allows the production of escape mutants in face of human immune system or antiviral therapies. HCV mutants with nucleotide substitutions either in B cell or in cytotoxic T lymphocyte (CTL) epitopes have been observed during chronic HCV infection (reviewed in Moorman et al., 2001; Rosenberg, 1999). Second, it facilitates the adaptation to new replication sites. For instance, the inventor found that HCV may adapt its replicative capability to a new host (the donor liver) by rapidly mutating the HVR1 domain in the setting of liver transplantation (Fan and Di Bisceglie, 2003). Third, it induces “original antigen sin” (OAS), a well-known immune phenomenon first described in influenza virus infection in 1950's (Fenner et al., 1974). OAS predicts weakened antibody responses in both concentration and affinity against HCV mutants, which facilitates the establishment of persistent HCV infection (Shimizu et al., 1994). OAS has also been observed in cellular immunity (Klenerman and Zinkernagel, 1998) and represents a major challenge in vaccine development for viruses with great genetic heterogeneity. Finally, because the dynamics of the immune response differ greatly for different HCV genotypes/subtypes (Yoshioka et al., 1997), it is difficult to select a vaccine strain.

There is accumulating evidence indicating that HCV infection does induce neutralizing antibodies, which play a partial role in the protection of HCV re-infection: (a) in patients with acute or chronic HCV infection, the natural resolution of HCV infection strongly correlates with the titers of putative neutralizing antibodies, anti-HVR1 (Ishii et al., 1998; Zibert et al., 1997); (b) chimpanzees inoculated with recombinant HCV E1/E2 heterodimer can be protected from experimental challenge with homologous virus (Choo et al., 1994). Putative neutralizing antibodies were detected in vaccinated chimpanzees but not in the control group (Lagging et al., 1998); (c) HCV-specific polyclonal globulins, purified from pools of human plasma that have high level of antibodies to HCV but normal ALT activities, may be capable of modifying the course of HCV infection and suppressing HCV replication in chimpanzees (Lemon et al., 2000), and post-exposure HCV immune globulin (HCIG) treatment also markedly prolonged the incubation period of acute hepatitis C (Krawczynski et al., 1996); and (d) in an epidemiological study with injecting drug users who are the high risk for HCV infection, the incidence of HCV infection was significantly lower in individuals with previous HCV infection than in people without previous HCV infection (Mehta et al., 2002).

More recently, with newly developed HCV neutralization assays, both genotype-dependent and cross-reactive neutralizing antibodies were detected in sera of patients with chronic HCV infection (Logvinoff et al., 2004). In face of such detectable neutralizing antibodies, HCV still establishes persistent infection, suggesting a possible deficiency of HCV neutralizing antibodies in quantity (titer and affinity) and/or quality (cross-protective capability to variants). The latter is perhaps the result of original antigen sin (OAS) as described above. Although many B cell epitopes have been mapped on HCV E1 and E2 (Habersetzer et al., 1998; Hadlock et al., 2000; Ishii et al., 1998; Jackson et al., 1997; Kato et al., 1994; Lee et al., 1999; Mink et al., 1994; Nakano et al., 1999; Rosa et al. 1996; Wang et al., 1996; Zhang et al., 1995; Zibert et al., 1997), all of them, including those epitopes presuming to have neutralizing potential, failed to induce neutralizing antibodies that can clear virus. These observations show that HCV does induce neutralizing antibodies in both genotype-dependent and genotype-independent patterns, which may not always be enough to clear the virus or prevent viral re-infection due to the deficiency in quality (cross-protective capability) and/or quantity (titer and affinity).

II. GENERATION OF ANCESTRAL HEPATITIS C VIRAL SEQUENCES

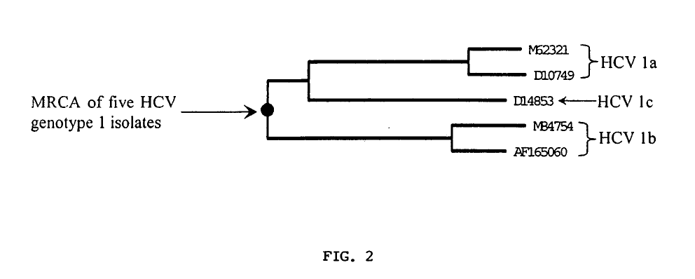

Pursuing ancestral characteristics of organisms or genes represents one of the most promising strategies for understanding complex biological or biomolecular functions. Unlike animals or plants, a virus evolves without traces of fossils. To reconstruct ancestral characteristics of viral genes, the only approach is to exploit nature's principles of Darwinian evolution, i.e., variation and selection, which can be simulated under a given nucleotide substitution model by mathematical models. Results are shown as phylogenies, in which end branches represent contemporary viral sequences while intra-nodes assume as ancestral nodes from which viruses start to diversify (FIG. 2). Ancestral sequences, also called most recent common ancestor (MRCA), can be inferred from ancestral nodes within a given phylogeny. It should be noted that a viral MRCA is different from its consensus sequence that is inferred based on the nucleotide frequency at each position over multiple viral sequences. The consensus sequence does not contain any evolutionary information.

Besides several technical issues such as the selection of a suitable nucleotide substitution model and the number of viral sequences used, the feasibility and reliability of ancestral reconstruction is largely dependent on the evolutionary mechanisms of the virus. Without drastic impairment of their replication capability, viruses experience adaptive evolution through changes of their genomes by three major mechanisms, i.e., point mutation including insertions and deletions, recombination and reassortment (Dolja and Carrington, 1992; Lai, 1992; Strauss and Strauss, 1988). HCV has a single viral genome with rare reports for its recombinationm and therefore evolves mainly through mutations. This is especially important in the reconstruction of ancestral characteristics of viral genes because recombination and/or ressortment result in evolutionary “jumps,” which make phylogenies less reliable. Ancestral HCV sequences may recover genetic information that is otherwise mutated during the long-term adaptive evolution. Recombinant proteins expressed with ancestral sequences may display protective epitopes that are highly immunodominant and conserved in all HCV genotypes/subtypes.

Biological characteristics of evolutionary ancestors have been studied for a few families of enzymes (Belinda et al., 2002; Jermann et al., 1995). However, such an approach has not been applied in vaccinology. Recently consensus sequences have been suggested in human immunodeficiency virus (HIV) vaccine development in which a high genetic heterogeneity is a major obstacle (Gaschen et al., 2002). Preliminary data showed that consensus HIV sequences induced broad neutralizing antibodies to many genotypes of HIV-1 (Gao et al., 2005). Artificial sequences created by other approaches, such as the DNA shuffling technique (Locher et al., 2005) have also been tested for improving vaccine antigens. However, all these strategies do not take into account the adaptive evolution, which is experienced by most, if not all, of microbes. Vaccine development by using ancestral sequences represents a novel approach for viruses with great genetic variability, and HCV is an outstanding candidate.

The present invention utilizes a method for developing an ancestral nucleotide sequence through reconstruction of phylogenetic trees. The ancestral nucleotide sequence may be directed to any one of myriad viruses or virus families, with HCV being exemplified herein. The method involves, generally, the steps of retrieving virus nucleic acid sequences from a genetic database (e.g., GenBank) and then editing and aligning those sequences using editing and alignment programs, which include for example Clustal W (Guindon and Gascuel, 2003), the BioEdit program available from North Carolina State University (available at www.mbio.ncsu.edu/BioEdit/bioedit.html), and the SegEd program available in the GCG package (Mehta et al., 2002). Any missing information, for example, the genotype for a given HCV sequence, may be determined by phylogenetic analyses, such as for example Molecular Evolutionary Genetics Analysis (MEGA; Holland et al., 1982). The sequences are then filtered to remove sequences that are below a particular size cut-off. Those remaining sequences that show signs of recombination are eliminated based on the detection of phylogenetic noise through split decomposition analysis (Di Bisceglie, 1998). These surviving sequences having greater than 99% identity at the nucleotide level are reduced to a single representative sequence. Model simulation and phylogenetic reconstruction are applied to the now remaining sequences. A preferred model is selected through a hierarchical likelihood ratio test (hLRT) simulated with the program Modeltest (Kato et al., 1992; Klenerman and Zinkernagel, 1998). A phylogenetic tree is then constructed by heuristic search using a maximum likelihood (ML) approach for separate or combined HCV genotypes. ML trees can be constructed using any one or more of known programs (e.g., PAUP, PHYML; Goldman, 1993; Krawczynski et al., 1996.) Once the trees are produced, the trees are then rooted respectively using following approaches: the strict or relaxed molecular clock model (Lai, 1992; Lee et al., 1999), non-reversible models of substitution, midpoint rooting, and outgroup criterion. (Gao et al., 2005; Higgins and Sharp, 1988; Lai, 1992; Lee et al., 1999; Logvinoff et al., 2004; Mink et al., 2004). The correctly rooted tree is then used as a template to simulate an ancestral sequence. Simulation of ancestral sequences at each internal node as well as the most recent common ancestor (MRCA) is conducted using an evolutionary program, such as for example the baseml program of the PAML package (Moorman et al., 2001). The ancestral sequence(s) are reconstructed at the nucleotide level.

III. HCV ENVELOPE PROTEINS

HCV encodes two envelope proteins, E1 and E2 (FIG. 1). The work described herein utilized 119 full-length HCV E1E2 sequences, having the GenBank accession numbers: AF011753, AF271632, AF290978, AF511948, AF511949, AF511950, AF529293, AJ278830, AJ557444, AY388455, AY615798, AY695436, AY695437, AY885238, AY958064, D10749, DQ061303, DQ061307, DQ061312, DQ061318, DQ061322, DQ061326, DQ061327, M62321, AB049087, AB049090, AB049093, AB049096, AB154186, AB154188, AB154192, AB154194, AB154198, AB154200, AB154202, AB154206, AF165056, AF176573, AF207758, AF207759, AF207761, AF207763, AF207764, AF207770, AF207773, AF356827, AF483269, AJ849974, AY070174, AY460204, D10934, D13406, D14484, D45172, D50480, D50481, D50484, D89815, D90208, L02836, L20498, M84754, M96362, U01214, U89019, X61592, AY051292, AY651061, D14853, AB030907, AB031663, AB047645, AF169002, AF169003, AF169004, AF169005, AF177036, AF238481, AF238482, AF238483, AF238484, AF238485, AF238486, AY232731, AY232733, AY232735, AY232737, AY232739, AY232741, AY232743, AY232745, AY232747, AY232749, AY746460, D00944, D10988, D50409, DQ155561, AF046866, AY958004, AY958024, AY958044, D17763, D28917, D49374, D63821, X76918, Y11604, AF064490, Y13184, AY859526, AY878650, D63822, D84262, D84263, D84264, D84265, DQ155560 and Y12083.

In mammalian cells transiently expressing HCV structural genes, E1 and E2 form a complex by non-covalent linkage and the proper folding of HCV E1 protein is dependent on E2 (Michalak et al., 1997; Reed and Rice, 2000; Spaete et al., 1992). In contrast, E2 folds independently of E1, suggesting a pivotal role for E2 in viral morphogenesis. E2 has a carboxy-terminal hydrophobic domain that appears to be inserted into the membrane of the endoplasmic reticulum (ER). Removal of this domain results in the production of a secreted soluble form of HCV E2 protein, indicating the existence of an ER retention signal within this domain (Cooquerel et al., 1998). Various C-terminal truncated forms of E2 protein have been investigated with respect to their folding and secretion. Michalak et al. (1997) found that HCV E2 protein truncated at amino acid 661 appeared to assume a more native structure than a longer version terminating at amino acid 715. E2 terminated at amino acid 661 was used to identify a putative HCV receptor, CD81 (Pileri et al., 1998). However, using a pseudotyped HIV/HCV system, Zhang et al. (2004) has demonstrated that the interaction between CD81 and truncated HCV E2 (soluble form) does not predict the CD81 dependence of HCV glycoprotein-mediated infection, indicating a more complex interaction between CD81 and pseudotyped HIV/HCV. Also, the infectivity of HCV pseudotype particles is dependent on the co-expression of HCV E1 and E2, which emphasizes different roles of E1 and E2 in mediating HCV entry into hepatocytes.

A. Fragments and Peptides of HCV Envelope Proteins

In addition to the use of full length sequences, the present invention contemplates the use of various peptides and truncated versions of HCV envelope proteins. For example, a portion of the protein as set forth in SEQ ID NOS:2 or 4 may be used in various embodiments of the invention. In certain embodiments, a fragment of the may comprise, but is not limited to about 5, about 6, about 7, about 8, about 9, about 10, about 11, about 12, about 13, about 14, about 15, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 450, about 500, about 600 or about 700 residues, and any range derivable therein.

It also will be understood that such partial sequences, along with full length HCV envelope proteins, may be joined or fused to additional residues, such as additional N— or C-terminal amino acids, and yet still be essentially as set forth in one of the sequences disclosed herein.

It is thus contemplated by the inventors that various changes may be made in the DNA or RNA sequences of genes or coding regions while retaining the same nucleic acid sequence. Table 1 shows the codons that encode particular amino acids.

TABLE 1
CODON TABLE

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

B. In vitro Production of HCV Polypeptides or Peptides

Various types of expression vectors are known in the art that can be used for the production of protein products. Following transfection with a expression vector, a cell in culture, e.g., a primary mammalian cell, a recombinant product may be prepared in various ways. A host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented (for exemplary methods see Freshney, 1992).

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth). Non-anchorage dependent or suspension cultures from continuous established cell lines are the most widely used means of large-scale production of cells and cell products. However, suspension cultured cells have limitations, such as tumorigenic potential and lower protein production than adherent cells. In further aspects of the invention, other protein production methods known in the art may be used, including but not limited to prokaryotic, yeast, and other eukaryotic hosts such as insect cells and the like.

Relatively small size peptides can be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides.

C. Protein Purification

It may be desirable to purify HCV envelope polypeptides and peptides. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, hydrophobic interaction chromatography, exclusion chromatography; polyacrylamide gel electrophoresis; isoelectric focusing. A particularly efficient method of purifying peptides is fast protein liquid chromatography (FPLC).

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, about 96%, about 97%, about 98%, about 99% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme.

IV. HCV ENVELOPE POLYNUCLEOTIDES

The term “gene” is used here to refer to the nucleic acid giving rise to a functional protein, polypeptide, or peptide-encoding unit, in this case an HCV envelope protein. In addition to the full length gene, which may contain non-coding sequences, the polynucleotides of the present invention contemplate shorter lengths that comprise less than all of a complete HCV polypeptide, including about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, or 540 or more nucleotides, nucleosides, or base pairs. Such sequences may be identical or complementary to all or part of SEQ ID NO:1 and SEQ ID NO:3, as well as SEQ ID NOS:5 and 6.

In particular embodiments, the invention concerns isolated nucleic acid segments and recombinant vectors incorporating DNA sequences that encode HCV envelope polypeptides or peptides. Such vectors used in the present invention, regardless of the length of the coding sequence itself, may be combined with other DNA or RNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

A. Vectors Encoding HCV Envelope Proteins

The present invention encompasses the use of vectors that encode all or part of one or more HCV envelope polypeptides. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). In particular embodiments, gene therapy or immunization vectors are contemplated. One of skill in the art would be well equipped to construct a vector through standard recombinant techniques, which are described in Maniatis et al. (1990) and Ausubel et al. (1996), both incorporated herein by reference.

The term “expression vector” or “expression construct” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” means that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the nucleic acid segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or exogenous, i.e., from a different source than the HCV envelope sequence. In some examples, a prokaryotic promoter is employed for use with in vitro transcription of a desired sequence. Prokaryotic promoters for use with many commercially available systems include T7, T3, and Sp6.

Table 2 lists several elements/promoters that may be employed, in the context of the present invention, to regulate the expression of a gene. This list is not intended to be exhaustive of all the possible elements involved in the promotion of expression but, merely, to be exemplary thereof. Table 3 provides examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Atchinson et al., 1986, |
| | 1987; Imler et al., 1987; Weinberger et al., |
| | 1984; Kiledjian et al., 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo |
| | et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-DRa | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Omitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion Molecule | |
| (NCAM) | |
| α₁-Antitrypain | Latimer et al., 1990 |
| H2B (TH2B) | Hwang et al., 1990 |
| Histone | |
| Mouse and/or Type | Ripe et al., 1989 |
| I Collagen | |
| Glucose-Regulated | Chang et al., 1989 |
| Proteins (GRP94 | |
| and GRP78) | |
| Rat Growth | Larsen et al., 1986 |
| Hormone | |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh |
| | et al., 1985; Firak et al., 1986; Herr et al., 1986; |
| | Imbra et al., 1986; Kadesch et al., 1986; Wang |
| | et al., 1986; Ondek et al., 1987; Kuhl et al., |
| | 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Chol et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987; Glue et al., 1988 |
| Hepatitis B Virus | Bulla et al., 1986; Jameel et al., 1986; Shaul et |
| | al., 1987; Spandau et al., 1988; Vannice et al., |
| | 1988 |
| Human | Muesing et al., 1987; Hauber et al., 1988; |
| Immunodeficiency | Jakobovits et al., 1988; Feng et al., 1988; |
| Virus | Takebe et al., 1988; Rosen et al., 1988; |
| | Berkhout et al., 1989; Laspia et al., 1989; |
| | Sharp et al., 1989; Braddock et al., 1989 |
| Cytomegalovirus | Weber et al., 1984; Boshart et al., 1985; |
| (CMV) | Foecking et al., 1986 |
| Gibbon Ape | Holbrook et al., 1987; Quinn et al., 1989 |
| Leukemia Virus | |

TABLE 3
Inducible Elements

| Element | Inducer | References |
|---|---|---|
| MT II | Phorbol Ester | Palmiter et al., 1982; |
| | (TFA) | Haslinger et al., 1985; Searle |
| | Heavy metals | et al., 1985; Stuart et al., |
| | | 1985; Imagawa et al., 1987, |
| | | Karin et al., 1987; Angel et |
| | | al., 1987b; McNeall et al., |
| | | 1989 |
| MMTV (mouse | Glucocorticoids | Huang et al., 1981; Lee et |
| mammary | | al., 1981; Majors et al., |
| tumor virus) | | 1983; Chandler et al., 1983; |
| | | Lee et al., 1984; Ponta et |
| | | al., 1985; Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2- | IL-6 | Kunz et al., 1989 |
| Macroglobulin | | |
| Vimentin | Serum | Rittling et al., 1989 |
| MHC Class I Gene | Interferon | Blanar et al., 1989 |
| H-2κb | | |
| HSP70 | ElA, SV40 Large | Taylor et al., 1989, 1990a, |
| | T Antigen | 1990b |
| Proliferin | Phorbol Ester- | Mordacq et al., 1989 |
| | TPA | |
| Tumor Necrosis | PMA | Hensel et al., 1989 |
| Factor | | |
| Thyroid | Thyroid Hormone | Chatterjee et al., 1989 |
| Stimulating | | |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki et al., 1998), D1A dopamine receptor gene (Lee et al., 1997), insulin-like growth factor II (Wu et al., 1997), human platelet endothelial cell adhesion molecule-1 (Almendro et al., 1996).

C. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

D. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999; Levenson et al., 1998; and Cocea, 1997; all incorporated herein by reference). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

E. Termination/Polyadenylation Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

F. Origins of Replication

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

G. Selectable and Screenable Markers

In certain embodiments of the invention, the cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

H. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector, expression of part or all of the vector-encoded nucleic acid sequences, or production of infectious viral particles. Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either an eukaryotic or prokaryotic host cell, particularly one that is permissive for replication or expression of the vector.

I. Expression Systems

Numerous expression systems exist that comprise at least all or part of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM from CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. The Tet-On™ and Tet-Off™ systems from CLONTECH® can be used to regulate expression in a mammalian host using tetracycline or its derivatives. The implementation of these systems is described in Gossen et al. (1992) and Gossen et al. (1995), and U.S. Pat. No. 5,650,298, all of which are incorporated by reference.

INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

J. Introduction of Nucleic Acids Into Cells

In certain embodiments, a nucleic acid may be introduce into a cell in vitro for production of polypeptides or in vivo for immunization purposes. There are a number of ways in which nucleic acid molecules such as expression vectors may be introduced into cells. In certain embodiments of the invention, the expression vector comprises an HCV infectious particle or engineered vector derived from an HCV genome. In other embodiments, an expression vector known to one of skill in the art may be used to express an HCV polypeptide. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986).

“Viral expression vector” is meant to include those vectors containing sequences of that virus sufficient to (a) support packaging of the vector and (b) to express a polynucleotide that has been cloned therein. In this context, expression may require that the gene product be synthesized. A number of such viral vectors have already been thoroughly researched, including adenovirus, adeno-associated viruses, retroviruses, herpesviruses, and vaccinia viruses.

Delivery may be accomplished in vitro, as in laboratory procedures for transforming cells lines, or in vivo or ex vivo, as in the treatment of certain disease states. One mechanism for delivery is via viral infection where the expression vector is encapsidated in an infectious viral particle. Several non-viral methods for the transfer of expression vectors into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), liposome (Ghosh and Bachhawat, 1991; Kaneda et al., 1989) and receptor-mediated transfection (Wu and Wu, 1987; Wu and Wu, 1988). Some of these techniques may be successfully adapted for in vivo or ex vivo use.

In certain embodiments, e.g., in vitro transformation of cells, the polynucleotide encoding an HCV gene may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression vector is delivered to a cell and where in the cell the nucleic acid remains is dependent on the type of expression vector employed.

V. VACCINES

Pharmaceutical compositions including HCV peptides and polypeptides will be formulated along the line of typical pharmaceutical drug and biological preparations. A discussion of formulations may be found in Remington's Pharmaceutical Sciences (1990). The percentage of active compound in any pharmaceutical preparation is dependent upon both the activity of the compound, in this case the ability of an HCV vaccine to stimulate an immune response against HCV infection. Typically, such compositions should contain at least 0.1% active compound. The percentage of the compositions and preparations may, of course, be varied and may conveniently be between about 2 to about 60% of the weight of the unit. The amount of active compounds in such therapeutically useful compositions is such that a suitable dosage will be obtained.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy injection is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, phenylmecuric nitrate, m-cresol, and the like. In many cases, it will be preferable to use isotonic solutions, for example, sugars or sodium chloride. Prolonged absorption of the injectable compositions can be brought about by the use in the compositions of agents delaying absorption, for example, aluminum monostearate, and gelatin.

Sterile injectable solutions are prepared by incorporating the active compounds in the required amount in the appropriate solvent with various of the other ingredients enumerated above, as required, followed by sterile filtration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques that yield a powder of the active ingredient plus any additional desired ingredient from a previously sterile-filtered solution thereof.

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

The active compounds of the present invention can be formulated for parenteral administration, e.g., formulated for injection via the intradermal, intravenous, intramuscular, intrathoracic, sub-cutaneous, or even intraperitoneal routes. Administration by the intradermal and intramuscular routes are specifically contemplated. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal, and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the age and possibly medical condition of the subject being treated. The person responsible for administration will, in any event, determine the appropriate dose for the individual subject.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Peptides or polypeptides may be administered in a dose that can vary from 0.01, 0.05, 0.1, 0.5, 1, 5, 10, 20, 30, 40, 50, 60, 70, 80, 90, 100 mg/kg of weight to 50, 60, 70, 80, 90, 100, 110, 120, 130, 140, 150, 160, 170, 180, 190, 200 mg/kg of weight in one or more daily, weekly, monthly, or yearly administrations during one or various days, weeks, months, or years. For a vaccine, the goal will be to develop a formulation that elicits protective immunity in as few doses as possible, hopefully a single dose. It is possible that booster doses will be required either for the primary immunization or for repeated immunization as the initial immune response wanes. The antigens or genes encoding antigens can be administered by parenteral injection (intravenous, intraperitoneal, intramuscular, subcutaneous, intracavity, intradermal or transdermic).

For viral vectors, one generally will prepare a viral vector stock of high titer. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient. Similar figures may be extrapolated for bacterial host delivery systems, or for liposomal or other non-viral formulations by comparing relative uptake efficiencies. Formulation as a pharmaceutically acceptable composition is discussed below.

In many instances, it will be desirable to have several or multiple administrations of the vaccine. The compositions of the invention may be administered 1, 2, 3, 4, 5, 6, 7, 8, 9, 10, or more times. The administrations will normally be at from one to twelve week intervals, more usually from one to four week intervals. Periodic re-administration will be desirable with recurrent exposure to the pathogen.

Protein vaccines often employ an immunogenic carrier protein, such as hepatitis B surface antigen, keyhole limpet hemocyanin or bovine serum albumin. Methods for performing this conjugation are well known in the art. Other immunopotentiating compounds are also contemplated for use with the compositions of the invention such as polysaccharides, including chitosan, which is described in U.S. Pat. No. 5,980,912, hereby incorporated by reference. The vaccine may further comprise an adjuvant, such as alum, Bacillus Calmette-Guerin, agonists and modifiers of adhesion molecules, tetanus toxoid, imiquinod, montanide, MPL, and QS21.

In a particular embodiment of the invention, the HCV vaccine may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991).

Current in vivo lipid delivery methods use subcutaneous, intradermal, intratumoral, or intracranial injection to avoid the toxicity and stability problems associated with cationic lipids in the circulation. The DOTAP:cholesterol lipid formulation is said to form a unique structure termed a “sandwich liposome.” This formulation is reported to “sandwich” DNA between an invaginated bi-layer or ‘vase’ structure. Beneficial characteristics of these lipid structures include a positive ρ, colloidal stabilization by cholesterol, two-dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990).

VI. MONITORING OF IMMUNE RESPONSES

In particular embodiments, the present invention concerns methods for detecting a immune response to HCV. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing HCV antibodies and contacting such a sample, under conditions permitting the formation of immunocomplexes, with HCV antigens or fragments thereof. The unwanted components will be washed, leaving the antigen immunocomplexed to the immobilized antibody. Such immunobinding methods also include methods for quantifying quantifying the amount of an antibody gen component in a sample and the detection and quantification of any immune complexes formed during the binding process. Here, one would obtain a sample suspected of containing an antigen, and contact the sample with an antibody against the ORF produced antigen, and then detect and quantify the amount of immune complexes formed under the specific conditions.

Contacting the chosen biological sample under effective conditions and for a period of time sufficient to allow the formation of immune complexes (primary immune complexes) is generally a matter of simply adding the composition to the antigen sample and incubating the mixture for a period of time long enough for the antibodies to form immune complexes with the HCV antigens. After this time, the mixture will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The HCV antigen employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Certain examples use antibody conjugates which a detecting antibody is linked to a detectable label. “Detectable labels” are compounds and/or elements that can be detected due to their specific functional properties, and/or chemical characteristics, the use of which allows the antibody to which they are attached to be detected, and/or further quantified if desired. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Enzymes (an enzyme tag) that will generate a colored product upon contact with a chromogenic substrate are also contemplated. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

VII. TREATMENT OF HCV INFECTION

The vaccines of the present invention may, in one embodiment, prevent infection by HCV or so limit infection and replication that there are no clinical symptoms. However, it may also be the case that symptoms are present in a vaccine recipient, though reduced in severity when compared to an unimmunized subject. In such situations, traditional HCV therapies may be provided to further limit or eliminate those remaining symptoms.

Interferon α-2b (IFN) can be used as monotherapy, but results in sustained response rates of only 10% to 25% of patients with HCV. Retreatment of primary non-responders with IFN alone is unsuccessful in most cases. Retreatment for relapsed patients with interferon α-2b in combination with ribavirin are very promising, but efficacy of combination therapy in primary non-responders is discussed controversially. Alternative approaches for non-responders might be high dose interferon induction therapy (10 MU QD) in combination with ribavirin or triple therapy with IFN, ribavirin and amantadine. Large trials are needed or already ongoing to confirm the efficacy and safety of these treatment options. PEGASYS™ (Roche) is a pegylated interferon which can be used as a monotherapy or with COPEGUS™, i.e., ribavirin. Milk thistle is an alternative medicine treatment that has some support in the mainstream medical community.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Inference of Ancestral HCV Envelope Sequences

Data compilation. Full-length HCV E1E2 sesquences were retrieved from the GenBank (www.ncbi.nlm.nih.gov/Genbank/index.html) and Los Alamos HCV database (hcv.lanl.gov) (Kuiken et al., 2005). Each sequence was manually examined to determine its genotype and exact length. Sequences were edited and aligned with Clustal W (Higgins and Sharp, 1988), BioEdit (www.mbio.ncsu.edu/BioEdit/bioedit.html) and SeqEd program in GCG package (Wisconsin GCG Package, Ver. 10.0). Missing genotype information for some sequences were determined by phylogenetic analyses with MEGA (Molecular Evolutionary Genetics Analysis) (Kumar et al., 2001) under neighbor-joining approach with kumar-2 parameter as nucleotide substitution model. The data was further filtered by excluding recombinants and sequences with high homogeneity in which only one of them was included. The final data set consists of 119 full-length HCV E1E2 sequences, designated number 1 to 119, respectively matching with their GenBank accession numbers AF011753, AF271632, AF290978, AF511948, AF511949, AF511950, AF529293, AJ278830, AJ557444, AY388455, AY615798, AY695436, AY695437, AY885238, AY958064, D10749, DQ061303, DQ061307, DQ061312, DQ061318, DQ061322, DQ061326, DQ061327, M62321, AB049087, AB049090, AB049093, AB049096, AB154186, AB154188, AB154192, AB154194, AB154198, AB154200, AB154202, AB154206, AF165056, AF176573, AF207758, AF207759, AF207761, AF207763, AF207764, AF207770, AF207773, AF356827, AF483269, AJ849974, AY070174, AY460204, D10934, D13406, D14484, D45172, D50480, D50481, D50484, D89815, D90208, L02836, L20498, M84754, M96362, U01214, U89019, X61592, AY051292, AY651061, D14853, AB030907, AB031663, AB047645, AF169002, AF169003, AF169004, AF169005, AF177036, AF238481, AF238482, AF238483, AF238484, AF238485, AF238486, AY232731, AY232733, AY232735, AY232737, AY232739, AY232741, AY232743, AY232745, AY232747, AY232749, AY746460, D00944, D10988, D50409, DQ155561, AF046866, AY958004, AY958024, AY958044, D17763, D28917, D49374, D63821, X76918, Y11604, AF064490, Y13184, AY859526, AY878650, D63822, D84262, D84263, D84264, D84265, DQ155560 and Y12083.

Model selection. The inventor then evaluated the most appreciate nucleotide substitution model for these 119 HCV sequences. This was done by using hierarchical likelihood ratio tests (hLRTs) that were simulated with the program Modeltest for total of 56 evolutionary models (Goldman, 1993; Posada and Crandall, 1998; Posada and Crandall, 2001). General time Reversible model (GTR) (Tavare) was selected together with among-site variation where proportion of invariable sites (I) and gamma distribution shape parameter (G) are 0.2188 and 0.6121, respectively.

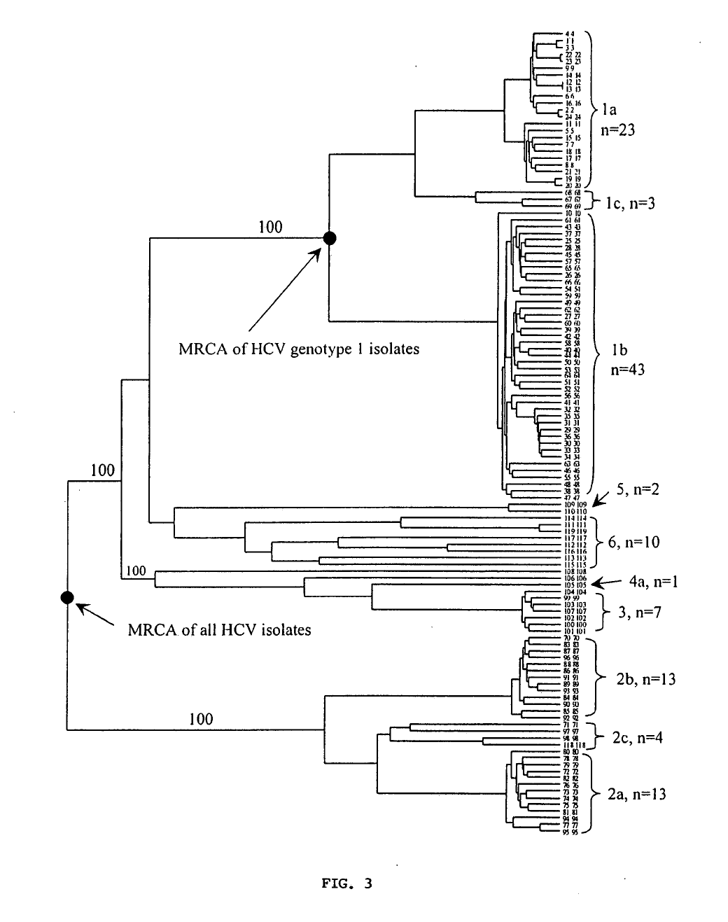

Reconstruction of phylogenetic trees. The best tree was recovered by heuristic search using maximum likelihood (ML) approach for the whole data set. The best-fit model and relative parameters described above were applied. All processes were completed with the program PAUP* (Swofford, PAUP Ver. 4.02b). Initially, it was failed to construct the tree with PAUP* directly due to the large numbers of sequences that assume unaffordable computation (years). As an alternative approach, the inventor first produced ML trees with PHYML program that implanted a simple hill-climbing algorithm for heuristic tree search and used a distance-based tree as a starting point (Guindon and Gascuel, 2003). The tree produced by PHYML was then transferred into PAUP* for further optimization and rooted by molecular clock approach (FIG. 3).

Inference of the ancestral HCV envelope sequence. The simulation of ancestral sequences at each internal node was done with “baseml” program in PAML package for both marginal and joint ancestral reconstruction (Yang, 1997). The tree shown in FIG. 3 served as the template. The inventor reconstructs ancestral sequences at nucleotide level rather than at codon or amino acid levels since the later two approaches ignore synonymous substitutions that may also experience positive selection (Novella et al., 2004). The inventor successfully inferred two ancestral sequences at the deepest and interior roots of the tree, representing the ancestors of all HCV isolates (HCVA1) and HCV genotype 1 isolates (HCVA2), respectively. Furthermore, the codon usages of HCVA1 and HCVA2 were optimized based on mammal species with program JCat (Grote et al., 2005) that implanted an algorithm for the calculation of the codon adaption index (CAI), a prevailing empirical measure of expressivity (Sharp and Li, 1987). The nucleotide sequences after codon optimization are designated as HCVA11 for HCVA1 and HCVA22 for HCVA2, respectively. All sequences are shown below.

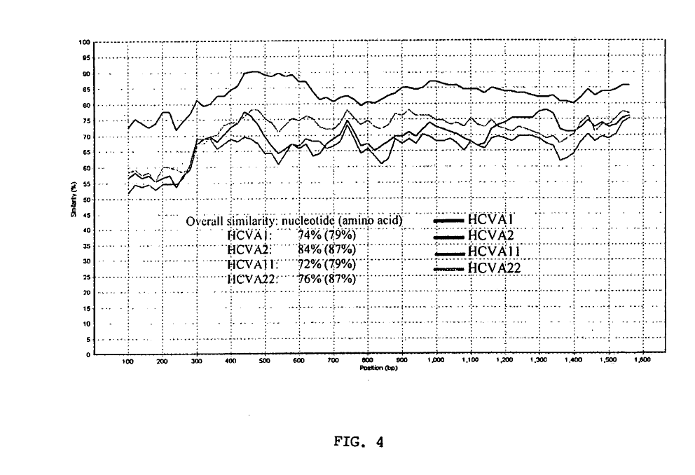

The similarity of ancestral HCV envelope sequences was examined at both nucleotide and amino acid levels using SimPlot program (Lole et al., 1999). HCVA1 and HCVA2 share 84% and 86% nucleotide and amino acid homogeneity, respectively. Interestingly, codon-optimized versions, HCVA11 and HCVA22 share 93% nucleotide homogeneity. The comparison of ancestral sequences and HCV Chiron strain (M62321) is shown in FIG. 4.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### X. REFERENCES

The following references, to the extent that they provide exemplary procedural or other details supplementary to those set forth herein, are specifically incorporated herein by reference.

U.S. Pat. No. 3,817,837, U.S. Pat. No. 3,850,752, U.S. Pat. No. 3,939,350, U.S. Pat. No. 3,996,345, U.S. Pat. No. 4,275,149, U.S. Pat. No. 4,277,437, U.S. Pat. No. 4,366,241, U.S. Pat. No. 4,683,202, U.S. Pat. No. 4,879,236, U.S. Pat. No. 5,650,298, U.S. Pat. No. 5,871,986, U.S. Pat. No. 5,925,565, U.S. Pat. No. 5,928,906, U.S. Pat. No. 5,935,819, U.S. Pat. No. 5,980,912.

Almendro et al., J. Immunol., 157(12):5411-5421, 1996.

Alter et al., N. Engl. J. Med., 341:556-562, 1999.

Angel et al., Mol. Cell. Biol., 7:2256, 1987.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Ausubel et al., In: Current Protocols in Molecular Biology, John, Wiley & Sons, Inc, New York, 1996.

Baichwal and Sugden, In: Gene Transfer, Kucherlapati (Ed.), NY, Plenum Press, 117-148, 1986.

Banerji et al., Cell, 27(2 Pt 1):299-308, 1981.

Banerji et al., Cell, 33(3):729-740, 1983.

Barany and Merrifield, In: The Peptides, Gross and Meienhofer (Eds.), Academic Press, NY, 1-284, 1979.

Belinda et al., Mol. Biol. Evol., 19:1483-1489, 2002.

Berkhout et al., Cell, 59:273-282, 1989.

Blanar et al., EMBO J., 8:1139, 1989.

Blight and Rice, J. Virol., 71:7345-7352, 1997.

Bodine and Ley, EMBO J., 6:2997, 1987.

Boshart et al., Cell, 41:521, 1985.

Bosze et al., EMBO J., 5(7):1615-1623, 1986.

Braddock et al., Cell, 58:269, 1989.

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Carbonelli et al., FEMS Microbiol. Lett., 177(1):75-82, 1999.

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chatterjee et al., Proc. Natl. Acad. Sci. USA, 86:9114, 1989.

Chen and Okayama, Mol. Cell Biol., 7(8):2745-2752, 1987.

Choi et al., Cell, 53:519, 1988.

Choo et al., Proc. Natl. Acad. USA, 91:1294-1298, 1994.

Choo et al., Science, 244:359-362, 1989.

Cocea, Biotechniques, 23(5):814-816, 1997.

Cocquerel et al., J. Virol., 72:2183-2191, 1998.

Cohen et al., J. Cell. Physiol., 5:75, 1987.

Costa et al., Mol. Cell. Biol., 8:81, 1988.

Cripe et al., EMBO J., 6:3745, 1987.

Culotta and Hamer, Mol. Cell. Biol., 9:1376, 1989.

Dandolo et al., J. Virology, 47:55-64, 1983.

De Jager et al., Semin. Nucl. Med., 23(2):165-179, 1993.

De Villiers et al., Nature, 312(5991):242-246, 1984.

Deschamps et al., Science, 230:1174-1177, 1985.

Di Bisceglie, Lancet., 351:351-355, 1998.

Dolja and Carrington, Semin. Virol., 3:315-326, 1992.

Doolittle and Ben-Zeev, Methods Mol Biol, 109:215-237, 1999.

Edbrooke et al., Mol. Cell. Biol., 9:1908, 1989.

Edlund et al., Science, 230:912-916, 1985.

Eigen, Scientific American, 269:42-49, 1993.

Fan and Di Bisceglie, J. Med. Virol., 70:212-218, 2003.

Fechheimer, et al., Proc Natl. Acad. Sci. USA, 84:8463-8467, 1987.

Feng and Holland, Nature, 334:6178, 1988.

Fenner et al., In: The Biology of Animal Viruses, 2^{nd} Ed., 417-418, Academic Press, London, 1974.

Firak and Subramanian, Mol. Cell. Biol., 6:3667, 1986.

Foecking and Hofstetter, Gene, 45(1):101-105, 1986.

Fraley et al., Proc. Natl. Acad. Sci. USA, 76:3348-3352, 1979.

Freshney, In: Animal Cell Culture, A Practical Approach, 2^{nd} Ed., Oxford Press, UK, 1992.

Fujita et al., Cell, 49:357, 1987.

Gabizon et al., Cancer Res., 50(19):6371-6378, 1990.

Gao et al., J. Virol., 79:1154-1163, 2005.

Gaschen et al., Science, 296:2354-2360, 2002.

Ghosh and Bachhawat, In: Liver Diseases, Targeted Diagnosis and Therapy Using Specific Receptors and Ligands, Wu et al. (Eds.), Marcel Dekker, NY, 87-104, 1991.

Gilles et al., Cell, 33:717, 1983.

Gloss et al., EMBO J., 6:3735, 1987.

Godbout et al., Mol. Cell. Biol., 8:1169, 1988.

Goldman, J. Mol. Evol., 36:182-198, 1993.

Goodbourn and Maniatis, Proc. Natl. Acad. Sci. USA, 85:1447, 1988.

Goodbourn et al., Cell, 45:601, 1986.

Gopal, Mol. Cell Biol., 5:1188-1190, 1985.

Gossen et al., Proc. Natl. Acad. Sci. USA, 89:5547-5551, 1992.

Gossen et al., Science, 268(5218):1766-1769, 1995.

Graham and Van Der Eb, Virology, 52:456-467, 1973.

Greene et al., Immunology Today, 10:272, 1989

Grosschedl and Baltimore, Cell, 41:885, 1985.

Grote et al., Nucleic Acid Res., 33:W526-531, 2005.

Guindon and Gascuel, Systematic Biology, 52:696-704, 2003.

Gulbis and Galand, Hum. Pathol., 24(12):1271-1285, 1993.

Habersetzer et al., Virology, 249:32-41, 1998.

Hadlock et al., J. Virol., 74:10407-10416, 2000.

Harland and Weintraub, J. Cell Biol., 101(3):1094-1099, 1985.

Haslinger and Karin, Proc. Natl. Acad. Sci. USA, 82:8572, 1985.

Hauber and Cullen, J. Virology, 62:673, 1988.

Hen et al., Nature, 321:249, 1986.

Hensel et al., Lymphokine Res., 8:347, 1989.

Herr and Clarke, Cell, 45:461, 1986.

Higgins and Sharp, Gene, 73:237-244, 1988.

Hijikata et al., Biochem. Biophys. Res. Commun., 175:220-228, 1991.

Hirochika et al., J. Virol., 61:2599, 1987.

Hirsch et al., Mol. Cell. Biol., 10:1959, 1990.

Holbrook et al., Virology, 157:211, 1987.

Holland et al., Science, 215:1577-1585, 1982.

Horlick and Benfield, Mol. Cell. Biol., 9:2396, 1989.

Huang et al., Cell, 27:245, 1981.

Hug et al., Mol. Cell. Biol., 8:3065, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Ishii et al., Hepatology, 28:1117-1120, 1998.

Jackson et al., J. Med. Virol., 51:67-79, 1997.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Jermann et al., Nature, 374:57-59, 1995.

Johnson et al., Mol. Cell. Biol., 9:3393, 1989.

Kadesch and Berg, Mol. Cell. Biol., 6:2593, 1986.

Kaneda et al., Science, 243:375-378, 1989.

Karin et al., Mol. Cell. Biol., 7:606, 1987.

Karin et al., Mol. Cell. Biol., 7:606, 1987.

Katinka et al., Cell, 20:393, 1980.

Kato et al., Biochem. Biophys. Res. Commun., 189:119-127, 1992.

Kato et al., J. Virol., 68:4776-4784, 1994.

Kawamoto et al., Mol. Cell. Biol., 8:267, 1988.

Kiledjian et al., Mol. Cell. Biol., 8:145, 1988.

Klamut et al., Mol. Cell. Biol., 10:193, 1990.

Klenerman and Zinkernagel, Nature, 394:482-485, 1998.

Koch et al., Mol. Cell. Biol., 9:303, 1989.

Kolykhalov, J. Virol., 70:3363-3371, 1996.

Kraus et al. FEBS Lett., 428(3):165-170, 1998.

Krawczynski et al., J. Infect. Dis., 173:822-828, 1996.

Kriegler and Botchan, In: Eukaryotic Viral Vectors, Gluzman (Ed.), Cold Spring Harbor: Cold Spring Harbor Laboratory, NY, 1982.

Kriegler and Botchan, Mol. Cell. Biol., 3:325, 1983.

Kriegler et al., Cell, 38:483, 1984.

Kriegler et al., Cell, 53:45, 1988.

Kuhl et al., Cell, 50:1057, 1987.

Kuiken et al., Bioinformatics, 21(3):379-384, 2005.

Kumar et al., Bioinformatics, 17:1244-1245, 2001.

Kunz et al., Nucl. Acids Res., 17:1121, 1989.

Lagging et al., J. Virol., 72:3539-3546, 1998.

Lai, Microbiol. Rev., 56:61-79, 1992.

Lareyre et al., J. Biol. Chem., 274(12):8282-8290, 1999.

Larsen et al., Proc Natl. Acad. Sci. USA., 83:8283, 1986.

Laspia et al., Cell, 59:283, 1989.

Latimer et al., Mol. Cell. Biol., 10:760, 1990.

Lee et al., Biochem. Biophys. Res. Commun., 240(2):309-313, 1997.

Lee et al., J. Virol., 73:11-18, 1999.

Lee et al., Nature, 294:228, 1981.

Lee et al., Nucleic Acids Res., 12:4191-206, 1984.

Lemon et al., Hepatology, 31:800-806, 2000.

Levenson et al., Hum. Gene Ther., 9(8):1233-1236, 1998.

Levinson et al., Nature, 295:79, 1982.

Lin et al., Mol. Cell. Biol., 10:850, 1990.

Locher et al., DNA & Cell Biology, 24:256-263, 2005.

Logvinoffet al., Proc. Natl. Acad. Sci. USA, 101:10149-10154, 2004.

Lole et al., J. Virol., 73:152-160, 1999.

Luria et al., EMBO J., 6:3307, 1987.

Lusky and Botchan, Proc. Natl. Acad Sci. USA, 83:3609, 1986.

Lusky et al., Mol. Cell. Biol., 3:1108, 1983.

Macejak and Sarnow, Nature, 353:90-94, 1991.

Majors and Varmus, Proc. Natl. Acad. Sci. USA, 80:5866, 1983.

Maniatis, et al., Molecular Cloning, A Laboratory Manual, Cold Spring Harbor Press, Cold Spring Harbor, N.Y., 1990.

McNeall et al., Gene, 76:81, 1989.

Mehta et al., Lancet., 359:1478-1483, 2002.

Merrifield, Science, 232(4748):341-347, 1986.

Michalak et al., J. Gen. Virol., 78:2299-2306, 1997.

Miksicek et al., Cell, 46:203, 1986.

Mink et al., Virology, 200:246-255, 1994.

Moorman et al., Archivum Immunologiae et Therapiae Experimentalis, 49:189-194, 2001.

Mordacq and Linzer, Genes and Dev., 3:760, 1989.

Moreau et al., Nucl. Acids Res., 9:6047, 1981.

Muesing et al., Cell, 48:691, 1987.

Nakamura et al., In: Handbook of Experimental Immunology (4^{th} Ed.), Weir et al. (Eds.), 1:27, Blackwell Scientific Publ., Oxford, 1987.

Nakano et al., J. Infect. Dis., 180:1328-1333, 1999.

National Institutes of Health Consensus Development Conference Panel, Hepatology, 26:2S-10S, 1997.

Neumann et al., Science, 282:103-107, 1998.

Ng et al., Nuc. Acids Res., 17:601, 1989.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nomoto et al., Gene, 236(2):259-271, 1999.

Novella et al., J. Mol. Biol., 342:1415-1421, 2004.

Ondek et al., EMBO J., 6:1017, 1987.

Ornitz et al., Mol. Cell. Biol., 7:3466, 1987.

Palmiter et al., Nature, 300:611, 1982.

Pech et al., Mol. Cell. Biol., 9:396, 1989.

Pelletier and Sonenberg, Nature, 334(6180):320-325, 1988.

Perez-Stable and Constantini, Mol. Cell. Biol., 10:1116, 1990.

Picard and Schaffner, Nature, 307:83, 1984.

Pileri et al., Science, 282:938-941, 1998.

Pinkert et al., Genes and Dev., 1:268, 1987.

Ponta et al., Proc. Natl. Acad. Sci. USA, 82:1020, 1985.

Porton et al., Mol. Cell. Biol., 10:1076, 1990.

Posada and Crandall, Syst. Biol., 50:580-601, 2001.

Posada Crandall, Bioinformatics, 14:817-818, 1998.

Potter et al., Proc. Natl. Acad. Sci. USA, 81:7161-7165, 1984.

Queen and Baltimore, Cell, 35:741, 1983.

Quinn et al., Mol. Cell. Biol., 9:4713, 1989.

Redondo et al., Science, 247:1225, 1990.

Reed and Rice, Current Topics in Microbiology & Immunology, 242:55-84, 2000.

Reisman and Rotter, Mol. Cell. Biol., 9:3571, 1989.

Remington's Pharmaceutical Sciences, 18th Ed. Mack Printing Company, pp. 1289-1329, 1990.

Resendez Jr. et al., Mol. Cell. Biol., 8:4579, 1988.

Ridgeway, In: Vectors: A Survey of Molecular Cloning Vectors and Their Uses, Rodriguez et al. (Eds.), Stoneham: Butterworth, 467-492, 1988.

Ripe et al., Mol. Cell. Biol., 9:2224, 1989.

Rippe, et al., Mol. Cell Biol., 10:689-695, 1990.

Rittling et al., Nuc. Acids Res., 17:1619, 1989.

Robertson et al., Arch. Virol., 143:2493-2501, 1998.

Rosa et al., Proc. Natl. Acad. Sci. USA, 3:1759-1763, 1996.

Rosen et al., Cell, 41:813, 1988.

Rosenberg, Gut., 44:759-764, 1999.

Sakai et al., Genes and Dev., 2:1144, 1988.

Sambrook et al., In: Molecular cloning, Cold Spring Harbor Laboratory Press, Cold Spring Harbor, N.Y., 2000.

Satake et al., J. Virology, 62:970, 1988.

Schaffner et al., J. Mol. Biol., 201:81, 1988.

Searle et al., Mol. Cell. Biol., 5:1480, 1985.

Sharp and Li, Nucleic Acid Res., 15:1281-1295, 1987.

Sharp and Marciniak, Cell, 59:229, 1989.

Shaul and Ben-Levy, EMBO J., 6:1913, 1987.

Sherman et al., Mol. Cell. Biol., 9:50, 1989.

Shimizu et al; J. Virol., 68:1494-1500, 1994.

Simmonds, Hepatology, 21:570-583, 1995.

Sleigh and Lockett, J. EMBO, 4:3831, 1985.

Spaete et al., Virology, 188:819-830, 1992.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Stewart and Young, In: Solid Phase Peptide Synthesis, 2d. ed., Pierce Chemical Co., 1984.

Strauss and Strauss, Annu. Rev. Microbiol., 42:657-683, 1988.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Swofford, PAUP*: Phylogenetic Analysis using Parsimony and Other Methods. Version 4.02b. Sinauer Associates. Sunderland, Mass.

Takebe et al., Mol. Cell. Biol., 8:466, 1988.

Tam et al., J. Am. Chem. Soc., 105:6442, 1983.

Tavare, In: Some mathematical questions in biology-DNA sequence analysis, 57-86, Miura (Ed.), Amer. Math. Soc., Providence, R.I.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (Ed.), NY, Plenum Press, 149-188, 1986.

Thiesen et al., J. Virology, 62:614, 1988.

Treisman, Cell, 42:889, 1985.

Tronche et al., Mol. Biol. Med., 7:173, 1990.

Trudel and Constantini, Genes and Dev., 6:954, 1987.

Tsumaki et al., J. Biol. Chem., 273(36):22861-22864, 1998.

Tur-Kaspa et al., Mol. Cell Biol., 6:716-718, 1986.

Tyndell et al., Nuc. Acids. Res., 9:6231, 1981.

Vannice and Levinson, J. Virology, 62:1305, 1988.

Vasseur et al., Proc Natl. Acad. Sci. USA, 77:1068, 1980.

Wang and Calame, Cell, 47:241, 1986.

Wang et al., J. Infec. Dis., 173:808-821, 1996.

Weber et al., Cell, 36:983, 1984.

Weinberger et al. Mol. Cell. Biol., 8:988, 1984.

Winoto and Baltimore, Cell, 59:649, 1989.

Wisconsin GCG package. Version 10.0. Oxford Molecular Group, Inc.

Wu and Wu, Biochemistry, 27: 887-892, 1988.

Wu and Wu, J. Biol. Chem., 262:4429-4432, 1987.

Wu et al., Biochem. Biophys. Res. Commun., 233(1):221-226, 1997.

Yamada et al., Virology, 223:255-261, 1996.

Yang et al. Proc. Natl. Acad. Sci. USA, 87:4144-4148, 1990.

Yang, Com. Appl. Biosci., 13:555-556., 1997

Yoshioka et al., J. Infect. Dis., 175:505-510, 1997.

Yutzey et al. Mol. Cell. Biol., 9:1397, 1989.

Zhang et al., J. Infect. Dis., 171:1356-1359, 1995.

Zhang et al., J. Virol., 78:1448-1455, 2004.

Zhao-Emonet et al., Biochim. Biophys. Acta, 1442(2-3):109-119, 1998.

Zibert et al., Hepatology, 25:1245-1249, 1997.

Zibert et al., J. Virol., 71:4123-417, 1997.

What is claimed:

1. A polynucleotide comprising a sequence that encodes a polypeptide comprising a sequence as set forth in SEQ ID NOS: 2 or 4.

2. The polynucleotide of claim 1, comprising the nucleotide sequence as set forth in SEQ ID NOS: 1, 3, 5 or 6.

3. A polynucleotide consisting essentially of a sequence that encodes a polypeptide comprising a sequence as set forth in SEQ ID NOS: 2 or 4.

4. The polynucleotide of claim 3, consisting essentially of a nucleotide sequence as set forth in SEQ ID NOS: 1, 3, 5, or 6.

5. A polynucleotide comprising a sequence that is greater than 81% identical to SEQ ID NOS: 1, 3, 5, or 6.

6. The polynucleotide of claim 5, wherein said sequence is greater than 84%, 86% or 88% identical to SEQ ID NOS: 1, 3, 5 or 6.

7. A polynucleotide comprising a nucleotide sequence that encodes a polypeptide comprising an amino acid sequence that is greater than 81% identical to SEQ ID NOS: 2 or 4.

8. The polynucleotide of claim 7, wherein said sequence is greater than 84%, 86% or 88% identical to SEQ ID NOS: 2 or 4.

9. A polypeptide comprising the amino acid sequence of SEQ ID NOS: 2 or 4.

10. An expression vector comprising a polynucleotide that encodes a polypeptide comprising a sequence as set forth in SEQ ID NOS: 2 or 4, wherein said polynucleotide is operably connected to a promoter.

11. The expression vector of claim 10, wherein the sequence is as set forth in SEQ ID NOS: 1, 3, 5, or 6.

12. The expression vector of claim 10, wherein the expression vector is a non-viral construct.

13. The expression vector of claim 10, wherein the expression vector is a viral construct.

14. The expression vector of claim 13, wherein said viral construct is an adenoviral construct.

15. A peptide comprising 10 to about 40 consecutive residues of SEQ ID NO: 2 or 4.

16. The peptide of claim 15, comprising 10 consecutive residues of SEQ ID NO: 2 or 4.

17. The peptide of claim 15, comprising 15 consecutive residues of SEQ ID NO: 2 or 4.

18. The peptide of claim 15, comprising 20 consecutive residues of SEQ ID NO: 2 or 4.

19. The peptide of claim 15, comprising 30 consecutive residues of SEQ ID NO: 2 or 4.

20. The peptide of claim 15, comprising 40 consecutive residues of SEQ ID NO: 2 or 4.

21. The peptide of claim 15, further comprising a non-HCV amino acid sequence.

22. The peptide of claim 21, wherein said non-HCV amino acid sequence is a peptide modifier of immunogenicity.

23. A method of inducing an immune response in a subject comprising administering to said subject a composition comprising a polypeptide comprising the amino acid sequence of SEQ ID NOS: 2 or 4, or an immunogenic fragment thereof.

24. The method of claim 23, wherein said subject is at risk of contracting HCV.

25. The method of claim 23, wherein said subject is a human.

26. The method of claim 23, wherein said composition further comprises an adjuvant.

27. The method of claim 23, further comprising administering said composition to said subject a second time.

28. The method of claim 21, further comprising assessing the immune response is said subject following administering.

29. A method of inducing an immune response in a subject comprising administering to said subject a composition comprising an expression construct comprising a polynucleotide encoding a polypeptide comprising the amino acid sequence of SEQ ID NOS: 2 or 4, or an immunogenic fragment thereof, wherein said polynucleotide is under the control of a promoter active in cells of said subject.

30. The method of claim 29, wherein said expression construct is a viral expression construct.


SEQUENCE LISTING (8 sequences)

SEQ ID NO: 1 - DNA, 1665 bp, Hepatitis C Virus
gtcgaataccgcaacgcctctggcgtctaccatgtcaccaatgactgccccaacaccagcatcgtgtatgaggccgaggacgtgatcctgcaccttcctggatgcgtgccctgtgtgagggagggcaatacgtcaaggtgctggataccagtcacacccaccgtggccgtcaggcacgccggcgcacccaccacggcgttccgaaggcacgtggatctgatcgtgggggccgctacattctgctcagctctgtacgtgggagatctgtgcggagccgtgttcctggtgggccaggtgttcaccttcaggcctcgccgccattggaccgtccaggactgcaactgttccatctacccaggccatattacaggccaccggatggcatgggacatgatgatgaactggtcgcctacaacgaccctggtggtggcccacgtgctgcggatcccccaggtcgtcctggacatgatcgccgggggccactggggcgtcctggtgggcatggcctacttctccatgcagggcaactgggccaaggtcatcgtcgtcctgttcctgtttgcaggggttgatgccaccacccacgccaccggcggcgccgcgggccgcgccgcccccggcctcgccggcctcttcacccccggggccaggcagaacatccagctcatcaacaccaatggcagctggcacatcaacaggaccgccctgaactgcaatgactccctaaagaccgggttcatcgcgggcctgttctacccccacaggttcaactcctcggggtgccccgagcggctggccagctgcaggcccctcgacgacttcggccaggggtggggccctttcaggtataacatcaccggcccctctgacgacaggccctactgctggcactacgcgcctagaccgtgtggcatcgtgccagcacggaatgtgtgtggcccagtgtactgcttcacccccagccctgtggtggtgggcaccaccgacaggaggggcgcccccacttacacctggggggagaatgagacagatgtgttcctgctgaacagcacgaggcccccgcggggcgcttggtttgggtgcacgtggatgaacggcacggggtttaccaagacgtgcggcgctccaccctgcaacatcaggggggacggcaacaacaacctgttgtgccccaccgattgcttcaggaagcacccagaggccacctacaccaagtgtgggtcagggccctggctcacacccaggtgcctggtcgactacccctacaggctgtggcattatccctgcactgtcaactacaccatcttcaaggtcaggatgtttgtagggggcgttgagcacaggttcgacgccgcgtgcaactggacccggggggagcgctgcgacctggaggacagggataggagcgagctgagcccgctgctgcattccaccacagagtgggccatcctgccctgctccttcacaaccatgcccgccctgtcaactggcctgatacacctccaccaaaacatcgtggacgtgcagtacctctatggcgttggctcggccatcgtatcgtgggccgtcaagtgggagtacgtcgtcctcgtcttcctgctcctggcagacgcaagcgtctgtgcctgcctgtggatgatgctgctgataggccaggccgaagca

SEQ ID NO: 2 - protein, 555 aa, Hepatitis C Virus
VEYRNASGVYHVTNDCPNTSIVYEAEDVILHLPGCVPCVREGNTSRCWIPVTPTVAVRHAGAPTTAFRRHVDLIVGAATFCSALYVGDLCGAVFLVGQVFTFRPRRHWTVQDCNCSIYPGHITGHRMAWDMMMNWSPTTTLVVAHVLRIPQVVLDMIAGGHWGVLVGMAYFSMQGNWAKVIVVLFLFAGVDATTHATGGAAGRAAPGLAGLFTPGARQNIQLINTNGSWHINRTALNCNDSLKTGFIAGLFYPHRFNSSGCPERLASCRPLDDFGQGWGPFRYNITGPSDDRPYCWHYAPRPCGIVPARNVCGPVYCFTPSPVVVGTTDRRGAPTYTWGENETDVFLLNSTRPPRGAWFGCTWMNGTGFTKTCGAPPCNIRGDGNNNLLCPTDCFRKHPEATYTKCGSGPWLTPRCLVDYPYRLWHYPCTVNYTIFKVRMFVGGVEHRFDAACNWTRGERCDLEDRDRSELSPLLHSTTEWAILPCSFTTMPALSTGLIHLHQNIVDVQYLYGVGSAIVSWAVKWEYVVLVFLLLADASVCACLWMMLLIGQAEA

SEQ ID NO: 3 - DNA, 1710 bp, Hepatitis C Virus
gtcgaagtgcgcaacgcctccggggtgtaccatgtcaccaatgattgccccaactcgagcattgtgtatgaggcagaggacatgatcatgcaccttcccgggtgtgtgccctgcgttcgggagggcaacgcctcgaggtgctgggtagcgctcactcccacggtcgccgccaggaacgccagcgtccccaccacggcgattcgacgtcacgtcgatctgcttgtcggggccgctgcattctgctcggctatgtacgtgggggacctgtgcggatctgtcttcctcgtcggccagctgttcaccttctcgcctcgccgccattggacagtacaggactgcaattgctccatctaccccggccatgtaacaggtcaccgcatggcttgggatatgatgatgaactggtcacctacaacagccctggtggtagcccagctgctccggatcccacaagccatcttggacatgatcgctggggcccactggggagtcctggcgggcatagcctacttctccatggtggggaactgggctaaggtcctggttgtgctgctgctatttgccggcgtcgacgcgaacacccacgtcaccgggggggctgcaggccgcaccacgtctggatttgctagcctcttcacacctggcgccaagcagaacatccagctcatcaacaccaacggcagctggcacatcaacaggactgccctgaactgcaatgactccctcaacaccgggttcgtagcagggctgttctactcccacaagttcaactcttcaggatgccccgagcggatggccagctgccgaccccttgacgacttcgaccaggggtggggccctatcagttacgccaacacaggcagctccgaccagaggccctactgctggcactacgcccctagaccgtgtggtattgtgccagcgaagaacgtgtgtggcccagtgtattgcttcacccccagccccgtggtggtgggaacgaccgacaggtcgggcgtccctacctacagctggggggagaatgagacggacgtgttcctcctcaacaacaccaggccgccgctgggcaattggttcggctgtacatggatgaactccactgggttcaccaagacgtgcggggcccccccttgtaacatcgggggggtcggcaacaacaccttgccctgccccactgattgcttccggaagcatccggaagccacatacactaagtgcggctcggggccttggcttacacccaggtgcctggtcgactacccatacaggctttggcattatccctgcaccgtcaactacaccatcttcaaggtcaggatgtttgtggggggcgttgagcacaggctcgacgctgcgtgcaactggactcggggagagcgttgcgacttggaggacagggataggtccgagctgagcccgctgctgctgtccaccacagagtggcagatcctgccctgttccttcacaaccctgccagccctgtccactggcctgatacatctccaccagaacatcgtggacgtgcagtacctgtacggtgtggggtcagccgttgtatcctgggccatcaagtgggagtacgtcgtgctcctcttcctgctgctggcagacgcgcgcatctgtgcctgcttgtggatgatgctactgatatcccaggcagaggcgtgtgcctgcttgtggatgatgctactgatatcccaggcagaggcg

SEQ ID NO: 4 - protein, 570 aa, Hepatitis C Virus
VEVRNASGVYHVTNDCPNSSIVYEAEDMIMHLPGCVPCVREGNASRCWVALTPTVAARNASVPTTAIRRHVDLLVGAAAFCSAMYVGDLCGSVFLVGQLFTFSPRRHWTVQDCNCSIYPGHVTGHRMAWDMMMNWSPTTALVVAQLLRIPQAILDMIAGAHWGVLAGIAYFSMVGNWAKVLVVLLLFAGVDANTHVTGGAAGRTTSGFASLFTPGAKQNIQLINTNGSWHINRTALNCNDSLNTGFVAGLFYSHKFNSSGCPERMASCRPLDDFDQGWGPISYANTGSSDQRPYCWHYAPRPCGIVPAKNVCGPVYCFTPSPVVVGTTDRSGVPTYSWGENETDVFLLNNTRPPLGNWFGCTWMNSTGFTKTCGAPPCNIGGVGNNTLPCPTDCFRKHPEATYTKCGSGPWLTPRCLVDYPYRLWHYPCTVNYTIFKVRMFVGGVEHRLDAACNWTRGERCDLEDRDRSELSPLLLSTTEWQILPCSFTTLPALSTGLIHLHQNIVDVQYLYGVGSAVVSWAIKWEYVVLLFLLLADARICACLWMMLLISQAEACACLWMMLLISQAEA

SEQ ID NO: 5 - DNA, 1665 bp, Hepatitis C Virus
gtggagtaccgcaacgccagcggcgtgtaccacgtgaccaacgactgccccaacaccagcatcgtgtacgaggccgaggacgtgatcctgcacctgcccggctgcgtgccctgcgtgcgcgagggcaacaccagccgctgctggatccccgtgacccccaccgtggccgtgcgccacgccggcgcccccaccaccgccttccgccgccacgtggacctgatcgtgggcgccgccaccttctgcagcgccctgtacgtgggcgacctgtgcggcgccgtgttcctggtgggccaggtgttcaccttccgcccccgccgccactggaccgtgcaggactgcaactgcagcatctaccccggccacatcaccggccaccgcatggcctgggacatgatgatgaactggagccccaccaccaccctggtggtggcccacgtgctgcgcatcccccaggtggtgctggacatgatcgccggcggccactggggcgtgctggtgggcatggcctacttcagcatgcagggcaactgggccaaggtgatcgtggtgctgttcctgttcgccggcgtggacgccaccacccacgccaccggcggcgccgccggccgcgccgcccccggcctggccggcctgttcacccccggcgcccgccagaacatccagctgatcaacaccaacggcagctggcacatcaaccgcaccgccctgaactgcaacgacagcctgaagaccggcttcatcgccggcctgttctacccccaccgcttcaacagcagcggctgccccgagcgcctggccagctgccgccccctggacgacttcggccagggctggggccccttccgctacaacatcaccggccccagcgacgaccgcccctactgctggcactacgccccccgcccctgcggcatcgtgcccgcccgcaacgtgtgcggccccgtgtactgcttcacccccagccccgtggtggtgggcaccaccgaccgccgcggcgcccccacctacacctggggcgagaacgagaccgacgtgttcctgctgaacagcacccgccccccccgcggcgcctggttcggctgcacctggatgaacggcaccggcttcaccaagacctgcggcgcccccccctgcaacatccgcggcgacggcaacaacaacctgctgtgccccaccgactgcttccgcaagcaccccgaggccacctacaccaagtgcggcagcggcccctggctgaccccccgctgcctggtggactacccctaccgcctgtggcactacccctgcaccgtgaactacaccatcttcaaggtgcgcatgttcgtgggcggcgtggagcaccgcttcgacgccgcctgcaactggacccgcggcgagcgctgcgacctggaggaccgcgaccgcagcgagctgagccccctgctgcacagcaccaccgagtgggccatcctgccctgcagcttcaccaccatgcccgccctgagcaccggcctgatccacctgcaccagaacatcgtggacgtgcagtacctgtacggcgtgggcagcgccatcgtgagctgggccgtgaagtgggagtacgtggtgctggtgttcctgctgctggccgacgccagcgtgtgcgcctgcctgtggatgatgctgctgatcggccaggccgaggcc

SEQ ID NO: 6 - protein, 555 aa, Hepatitis C Virus; identical to SEQ ID NO: 2

SEQ ID NO: 7 - DNA, 1665 bp, Hepatitis C Virus
gtggaggtgcgcaacgccagcggcgtgtaccacgtgaccaacgactgccccaacagcagcatcgtgtacgaggccgaggacatgatcatgcacctgcccggctgcgtgccctgcgtgcgcgagggcaacgccagccgctgctgggtggccctgacccccaccgtggccgcccgcaacgccagcgtgcccaccaccgccatccgccgccacgtggacctgctggtgggcgccgccgccttctgcagcgccatgtacgtgggcgacctgtgcggcagcgtgttcctggtgggccagctgttcaccttcagcccccgccgccactggaccgtgcaggactgcaactgcagcatctaccccggccacgtgaccggccaccgcatggcctgggacatgatgatgaactggagccccaccaccgccctggtggtggcccagctgctgcgcatcccccaggccatcctggacatgatcgccggcgcccactggggcgtgctggccggcatcgcctacttcagcatggtgggcaactgggccaaggtgctggtggtgctgctgctgttcgccggcgtggacgccaacacccacgtgaccggcggcgccgccggccgcaccaccagcggcttcgccagcctgttcacccccggcgccaagcagaacatccagctgatcaacaccaacggcagctggcacatcaaccgcaccgccctgaactgcaacgacagcctgaacaccggcttcgtggccggcctgttctacagccacaagttcaacagcagcggctgccccgagcgcatggccagctgccgccccctggacgacttcgaccagggctggggccccatcagctacgccaacaccggcagcagcgaccagcgcccctactgctggcactacgccccccgcccctgcggcatcgtgcccgccaagaacgtgtgcggccccgtgtactgcttcacccccagccccgtggtggtgggcaccaccgaccgcagcggcgtgcccacctacagctggggcgagaacgagaccgacgtgttcctgctgaacaacacccgcccccccctgggcaactggttcggctgcacctggatgaacagcaccggcttcaccaagacctgcggcgcccccccctgcaacatcggcggcgtgggcaacaacaccctgccctgccccaccgactgcttccgcaagcaccccgaggccacctacaccaagtgcggcagcggcccctggctgaccccccgctgcctggtggactacccctaccgcctgtggcactacccctgcaccgtgaactacaccatcttcaaggtgcgcstgttcgtgggcggcgtggagcaccgcctggacgccgcctgcaactggacccgcggcgagcgctgcgacctggaggaccgcgaccgcagcgagctgagccccctgctgctgagcaccaccgagtggcagatcctgccctgcagcttcaccaccctgcccgccctgagcaccggcctgatccacctgcaccagaacatcgtggacgtgcagtacctgtacggcgtgggcagcgccgtggtgagctgggccatcaagtgggagtacgtggtgctgctgttcctgctgctggccgacgcccgcatctgcgcctgcctgtggatgatgctgctgatcagccaggccgaggcc

SEQ ID NO: 8 - protein, 555 aa, Hepatitis C Virus
  misc_feature (440)..(440): Xaa can be any naturally occurring amino acid
VEVRNASGVYHVTNDCPNSSIVYEAEDMIMHLPGCVPCVREGNASRCWVALTPTVAARNASVPTTAIRRHVDLLVGAAAFCSAMYVGDLCGSVFLVGQLFTFSPRRHWTVQDCNCSIYPGHVTGHRMAWDMMMNWSPTTALVVAQLLRIPQAILDMIAGAHWGVLAGIAYFSMVGNWAKVLVVLLLFAGVDANTHVTGGAAGRTTSGFASLFTPGAKQNIQLINTNGSWHINRTALNCNDSLNTGFVAGLFYSHKFNSSGCPERMASCRPLDDFDQGWGPISYANTGSSDQRPYCWHYAPRPCGIVPAKNVCGPVYCFTPSPVVVGTTDRSGVPTYSWGENETDVFLLNNTRPPLGNWFGCTWMNSTGFTKTCGAPPCNIGGVGNNTLPCPTDCFRKHPEATYTKCGSGPWLTPRCLVDYPYRLWHYPCTVNYTIFKVRXFVGGVEHRLDAACNWTRGERCDLEDRDRSELSPLLLSTTEWQILPCSFTTLPALSTGLIHLHQNIVDVQYLYGVGSAVVSWAIKWEYVVLLFLLLADARICACLWMMLLISQAEA